(19) United States
(12) Patent Application Publication
(10) Pub. No.: US 2026/0272718 A1
(43) Pub. Date: Sep. 17, 2026

(54) FILM-TYPE PATCH AND METHOD OF MANUFACTURING SAME

(71) Applicant: DOCTORS YOUNG AND YOUNG INCORPORATED, Seoul (KR)

(72) Inventors: Ji Yung YUN, Yongin-si (KR); Jin Ho KIM, Yongin-si (KR); Kwang Su KIM, Seoul (KR)

(73) Assignee: DOCTORS YOUNG AND YOUNG INCORPORATED, Seoul (KR)

(21) Appl. No.: 19/473,304

(22) PCT Filed: Apr. 9, 2024
(86) PCT No.: PCT/KR2024/004683

§ 371 Date: Oct. 7, 2025

(30) Foreign Application Priority Data
Apr. 10, 2023 (KR) 10-2023-0046709

(51) Int. Cl. A61F 13/00; A61F 13/02; A61K 9/70

(52) U.S. Cl. CPC A61F 13/00063; A61F 13/00995; A61F 13/0233; A61K 9/7092; A61F 2013/00604; A61F 2013/00646; A61F 2013/00859; A61F 2013/00906

(57) ABSTRACT

The present invention provides a film-type patch containing a support layer, an adhesive layer laminated on an upper surface of the support layer, and a plurality of patterned protrusions spaced apart from each other and attached to an upper surface of the adhesive layer, wherein the patterned protrusions contain a skin penetrating substance, the space between the protrusions is provided with a number of holes penetrating the support layer and the adhesive layer, and a method of manufacturing the same.

### TECHNICAL FIELD

This application claims the benefit of priority based on Korean Patent Application No. 10-2023-0046709 filed on Apr. 10, 2023, the entire contents of which are incorporated as part of this specification.

The present invention relates to a film-type patch and a method of manufacturing the same.

### BACKGROUND ART

The skin is composed of stratum corneum, epidermis, dermis, and subcutaneous tissue from the outer surface. The epidermis is a natural ‘protective membrane’ that protects the human body from the outside, but it acts as a natural ‘barrier’ when administering ingredients such as drugs through the skin. For example, when delivering bioactive materials with effects such as wrinkle improvement, whitening, and fat decomposition through the skin, these materials must be sufficiently delivered to the dermis, but since the skin acts as a barrier, such delivery is very difficult. Therefore, the skin penetration rate of these bioactive materials is very low at around 0.5 to 3.0%.

In addition, bioactive materials as described above have particle size larger than the gap between epidermal cells of the skin, making it difficult to pass through the epidermis. Therefore, to overcome these limitations, nanotechnology is being actively researched to manufacture and use bioactive materials in nano sizes.

As the nanotechnology described above, emulsion technology is being applied, and recently, nano-emulsion technology that forms an emulsion with a size of 50 to 500 nm has been developed, and its application is becoming active. In particular, with the development of such nanotechnology, the technology for delivering bioactive materials through patches attached to the skin as a non-invasive method is also gradually developing. The use of such patches is known as a promising approach for increasing the skin's permeability to drugs.

As a method for delivering a bioactive material by a patch, a method of forming drug-containing protrusions on the patch and creating microchannels in the skin through these protrusions is known. The protrusions are generally manufactured using a molding method, and the molds are prepared using complex microfabrication procedures in a clean room. These procedures include photolithography using deep X-ray lithography and ultraviolet (UV) lithography of LIGA (Lithographie, Galvanoformung, Abformung). However, these methods have the disadvantage of being very time-consuming, requiring advanced clean room facilities, and excessively increasing the manufacturing cost. For example, the protrusions are generally formed in the shape of an acrosome, and this type of needles have a small area of contact with the skin, so they cannot effectively and quickly deliver drugs, and in particular, when applied to a wound, they cause problems by irritating the wound and hindering the healing effect.

In addition, in the case of a patch formed with protrusions as described above, there is a disadvantage in that the adhesive strength to the skin is weakened. That is, since the adhesive surface of the patch is lifted from the skin by the protrusions, and the area of the adhesive layer is reduced by the formed area of the protrusions, these patches face a difficult challenge related to improving adhesive strength to the skin.

Meanwhile, cosmetics and ointments containing nano-materials have been developed and are actually being applied to the skin. However, when applied to the skin, a large amount of nano-materials evaporates or aggregates due to external conditions (sunlight (including UV), heat, natural evaporation, wind, etc.), and thus the amount of nano-medicine delivered to the skin is considerably small compared to the actual administered amount. Accordingly, considering these circumstances, since it is required to apply expensive nano-materials in excessive amounts, it acts as a cause of the increase in the price of the product. However, if these nano-materials are used through patches, significant effects can be achieved in terms of cost and effectiveness.

### PRIOR ART DOCUMENT

### Patent Document

(Patent Document 1) Korean Patent No. 10-2369762

### DISCLOSURE

### Technical Problem

The present invention is intended to solve the problems described above in the prior art, and
it is an object of the present invention to provide a film-type patch that can significantly improve adhesive strength to the skin by forming a plurality of holes penetrating a substrate and an adhesive layer, can increase the contact area between protrusions and skin to increase the drug delivery speed, can effectively deliver a drug over a wide area without irritating the wound when attached to a wound, and can significantly reduce the number of manufacturing processes and thus greatly reduce the manufacturing cost, and a method of manufacturing the same.

In addition, it is another object of the present invention to provide a film-type patch having excellent productivity and quality by preventing the protrusions from being detached from the adhesive layer during punching, and a method of manufacturing the same.

### Technical Solution

In order to achieve the objects described above, the present invention provides a film-type patch comprising,
a support layer,
an adhesive layer laminated on an upper surface of the support layer, and
a plurality of patterned protrusions spaced apart from each other and attached to an upper surface of the adhesive layer,
wherein the patterned protrusions contain a skin penetrating substance, and
the space between the protrusions is provided with a number of holes penetrating the support layer and the adhesive layer.

In addition, the present invention provides a method of manufacturing the film-type patch of the present invention described above, comprising the steps of:
a) laminating a protrusion forming sheet on one side of an adhesive sheet;
b) punching out a plurality of patterned protrusions into the protrusion forming sheet; and
c) removing the protrusion forming sheet excluding the protrusions patterned by punching from the adhesive sheet,
wherein before or after step (a), or before or after step (c), a support substrate is laminated on the other side of the adhesive sheet, and then the adhesive sheet and the support substrate located in the space between the protrusions are perforated to form a plurality of holes.

In addition, the present invention provides a method of manufacturing the film-type patch of the present invention described above, comprising the steps of:
a) laminating a protrusion forming sheet on one side of a first adhesive sheet;
b) punching out a plurality of patterned protrusions into the protrusion forming sheet;
c) removing the protrusion forming sheet excluding the protrusions patterned by punching from the first adhesive sheet;
d) laminating a second adhesive sheet on the upper surface of the patterned protrusions; and
e) separating the second adhesive sheet and the protrusions adhered thereto from the first adhesive sheet,
wherein, before or after step d), or after step (e), a support substrate is laminated on the other side of the second adhesive sheet, and then the adhesive sheet and the support substrate located in the space between the protrusions are perforated to form a plurality of holes.

### Advantageous Effects

The film-type patch of the present invention provides:
an effect of greatly improving the adhesive strength to the skin by having a plurality of holes penetrating the substrate and the adhesive layer; and
significantly increases the contact area between the protrusions and the skin by including the patterned protrusions, and thus increases the drug delivery speed, and when attached to a wound, unlike the conventional acrosome-shape needle, provides the effect of effectively delivering the drug through a wide area without irritating the wound.

In addition, since the patterned protrusions are directly bonded to the adhesive layer without a separate support, they provide the effect of significantly increasing the area of the adhesive layer in contact with the skin compared to conventional patches using a separate support.

In addition, by controlling the content of the solvent contained in the protrusions, the protrusions are prevented from being detached from the adhesive layer during punching, thereby providing excellent productivity and quality.

The method of manufacturing the film-type patch of the present invention is very simple compared to conventional methods and provides high productivity, since protrusions are easily formed by a punching process. In addition, due to these effects, it provides the effect of significantly reducing the production cost of the patch.

### DESCRIPTION OF DRAWINGS

FIG. 1 is a cross-sectional view schematically illustrating one embodiment of the film-type patch of the present invention.

FIG. 2 is a plan view schematically illustrating one embodiment of the film-type patch of the present invention.

FIG. 3 is a cross-sectional view schematically illustrating another embodiment of the film-type patch of the present invention.

FIG. 4 is a photograph of another embodiment of the film-type patch of the present invention.

FIG. 5 is a photograph of another embodiment of the film-type patch of the present invention, in which the patch is in an attached state.

FIG. 6 is a photograph of the attached state of the film-type patches of Example 1 of the present invention and Comparative Example 1.

FIG. 7 is a photograph of the attached state of the film-type patch of Comparative Example 2 of the present invention.

FIGS. 8 and 9 are drawings schematically illustrating one embodiment of a method of manufacturing a film-type patch of the present invention.

FIGS. 10 and 11 are drawings schematically illustrating another embodiment of a method of manufacturing a film-type patch of the present invention.

FIG. 12 is a photograph of the shape of a film-type patch manufactured in Example 1 of the present invention.

FIG. 13 is a photograph of the shape of a film-type patch manufactured in Example 2 of the present invention.

FIG. 14 is a photograph of the shape of a film-type patch manufactured in Comparative Example 3.

FIG. 15 is a photograph of the shape of a film-type patch manufactured in Comparative Example 4.

FIG. 16 is a photograph showing one embodiment of a perforating needle used to form a hole penetrating a support layer and an adhesive layer in the present invention.

### BEST MODE

Hereinafter, embodiments of the present invention will be described in detail with reference to the attached drawings so that those skilled in the art can easily implement the present invention. However, the present invention can be implemented in many different forms and is not limited to the embodiments set forth herein. Throughout the specification, similar parts are designated by the same drawing reference numerals.

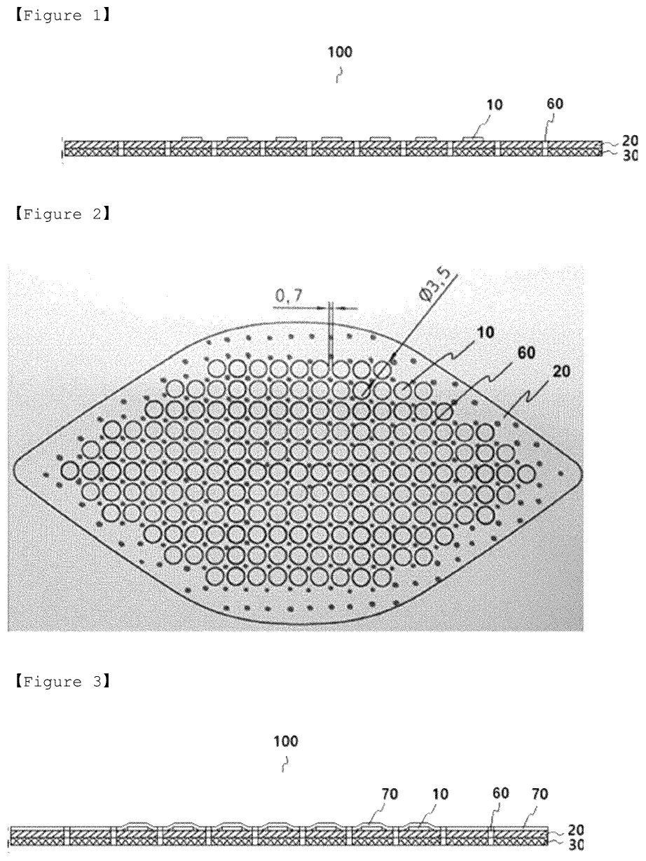

FIG. 1 is a cross-sectional view schematically illustrating one embodiment of the patch according to the present invention. As illustrated in FIG. 1, the patch 100 of the present invention comprises a support layer 30, an adhesive layer 20 laminated on an upper surface of the support layer, and a plurality of patterned protrusions 10 spaced apart from each other and attached to one surface of the adhesive layer 20,
wherein a plurality of holes 60 penetrating the support layer 30 and the adhesive layer 20 are provided in the space between the protrusions 10.

Since the film-type patch 100 of the present invention includes protrusions 10, its adhesiveness to the skin may be weakened compared to a patch that does not include protrusions. Therefore, the present invention has a structure that enhances adhesiveness by forming a plurality of holes 60 penetrating the support layer 30 and the adhesive layer 20 in the space between the protrusions 10 as described above. When the holes 60 are formed only in the support layer 30, the effect of improving the adhesiveness to the skin is insufficient, whereas when a plurality of holes are formed penetrating the support layer 30 and the adhesive layer 20, the adhesiveness is greatly improved.

In particular, when a patch is attached to the skin, the adhesiveness of the patch is significantly reduced by foreign substances such as oil or sweat discharged from the skin or moisture, and a lifting phenomenon occurs in the early stage of attachment. On the other hand, in the case of the film-type patch of the present invention, a plurality of holes is formed through the support layer 30 and the adhesive layer 20, so that foreign substances such as oil or sweat or moisture can be discharged out of the patch through the holes, thereby providing the effect of significantly increasing the adhesiveness of the patch.

The film-type patch of the present invention can be attached to, for example, the chin/neck area, and in this case, wrinkles are formed and then disappeared repeatedly on the skin according to the movement of the skin. Accordingly, when a patch with a general structure is attached, the adhesive state of the patch is not maintained for a long time, and the patch detaches from the skin within a short period of time. On the other hand, in the case of the patch of the present invention in which a plurality of holes is formed, even if wrinkles are repeatedly formed and then disappeared on the skin according to the movement of the skin in areas such as the chin/neck, the effect of maintaining the adhesive state for a long time is provided.

In one embodiment of the present invention, the holes 60 may be provided in 2 to 8 pieces in a space formed between one protrusion and other adjacent protrusions.

The hole may have a longitudinal length of 0.1 mm to 2.5 mm, preferably 0.5 mm to 2 mm, and more preferably 0.5 mm to 1.5 mm. If the longitudinal length of the hole is less than 0.5 mm, it is difficult to form the hole and the effect of improving the adhesiveness to the skin due to the hole is also reduced, which is not desirable. In addition, if the longitudinal length of the hole exceeds 2.5 mm, the skin adhesion area of the adhesive layer is significantly reduced, which reduces the effect of improving the adhesiveness to the skin, and thus is not desirable.

The short direction length of the hole may be 0.5 mm to 2.5 mm.

The hole may be formed in a circular, oval, or polygonal shape, etc. but is not limited thereto, and is not particularly limited as long as it has a shape that can improve the flexibility and adhesiveness of the film-type patch.

In one embodiment of the present invention, a plurality of holes penetrating the support layer and the adhesive layer may be further formed in a portion of the entire area of the film-type patch where the protrusions are not formed, as illustrated in FIG. 2. In that case, the size of the holes, the spacing between the holes and the arrangement thereof may be the same as or different from the portion in which the protrusions are formed.

In one embodiment of the present invention, the hole can be formed, for example, using a perforator, but is not limited thereto, and various methods known in the art or conventional hole forming methods can be applied without limitation.

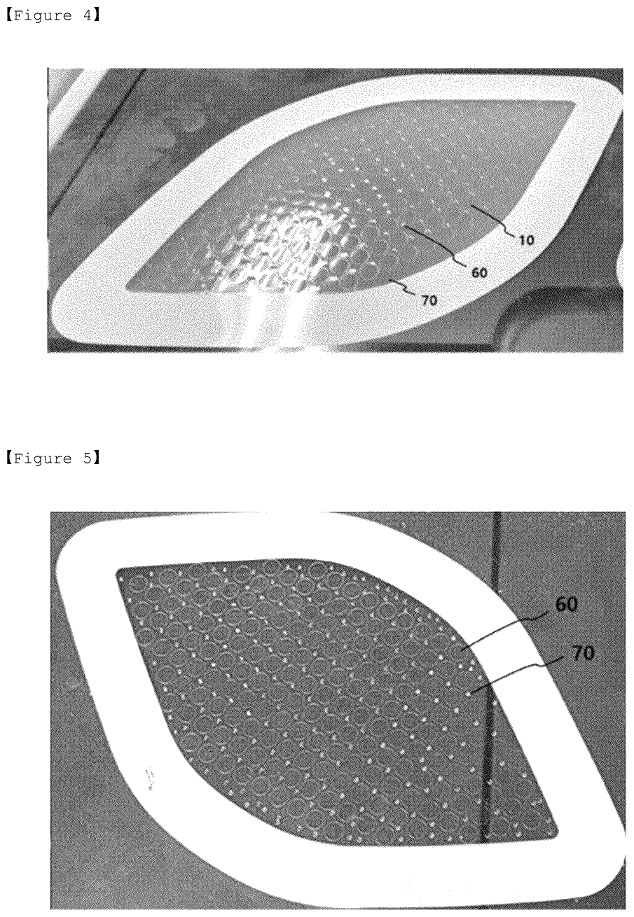

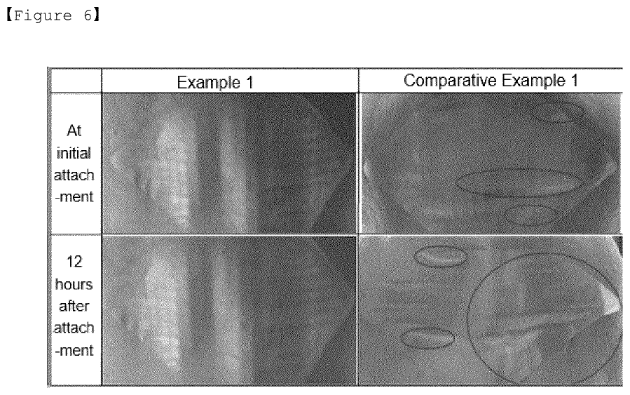

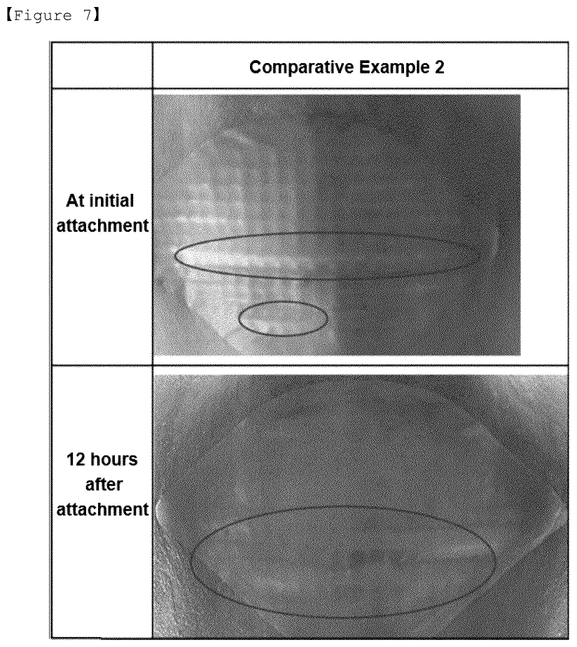

In one embodiment of the present invention, the film-type patch 100 may further include a releasing film 70 laminated on an exposed surface of the adhesive layer 20 and a plurality of patterned protrusions 10 spaced apart from each other and attached to one surface of the adhesive layer 20, as shown in FIGS. 3 and 4.

As the releasing film 70, a polymer film generally used in this field can be used. For example, a PET film can be used as the releasing film, but is not limited thereto.

The releasing film 70 is removed when the film-type patch 100 is attached to the skin, and functions to protect the surfaces of the adhesive layer and the protrusions from foreign substances and prevent the solvent from evaporating from the surfaces of the adhesive layer and the protrusions.

In one embodiment of the present invention, when the film-type patch 100 is further provided with the releasing film 70, as shown in FIGS. 3 and 4, a plurality of holes 60 penetrating the support layer 30 and the adhesive layer 20 may be formed to also penetrate the releasing film 70. That is, the plurality of holes may be formed to penetrate the support layer 30, the adhesive layer 20, and the releasing film 70.

As described above, when a plurality of holes 60 are formed while penetrating the support layer 30, the adhesive layer 20, and the releasing film 70, it can be expected that the shape of the hole is better maintained. That is, the hole formed in the adhesive layer 20 of the film-type patch 100 may be narrowed over time due to the physical properties of the adhesive. However, when the hole is formed in the releasing film 70, the hole formed in the adhesive layer is suppressed from narrowing, so that the shape of the initially perforated hole is easily maintained. This effect appears to be because the shape of the hole penetrating the support substrate 30, the adhesive layer 20, and the releasing film 70 is supported on both sides by the hole formed in the support substrate 30 and the releasing film 70.

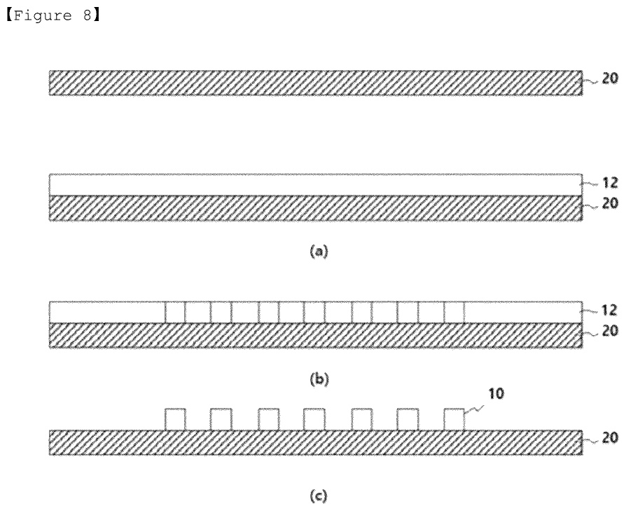

The effect described above is well confirmed by FIG. 5. That is, FIG. 5 is a photograph of the film-type patch in the attached state after manufacturing the film-type patch in which a plurality of holes is formed to penetrate the support layer 30, the adhesive layer 20, and the releasing film 70 and removing the releasing film 70 30 days later. From FIG. 5, it can be confirmed that even after 30 days from the formation of the hole, the initially formed hole was still maintained in the attached film-type patch.

Since the patch of the present invention has patterned protrusions 10 directly attached to the adhesive layer 20 without a support, the adhesive layer, which was covered by the support in the conventional patch, is revealed, thereby additionally improving the adhesiveness of the patch. That is, an adhesive surface exists not only at both ends of the patch but also at the center, and accordingly, the area of the adhesive layer attached to the skin is significantly expanded compared to the conventional technology of forming protrusions on a support. As the area of the adhesive layer increases in this way, it is possible for the protrusions 10 to maintain a more solid bond with the skin.

In addition, for this reason, even if the surface to which it is attached is curved or the object to which it is attached moves, it can maintain a much better attachment state than the conventional technology.

In one embodiment of the present invention, the protrusions may be formed in the shape of, for example, cylinders, elliptical columns, or polygonal columns, but are not limited thereto. When the protrusion is formed in a columnar shape, the surface in contact with the skin becomes a plane shape, so the contact area between the protrusion and the skin is greatly increased, and accordingly, the transfer speed of the skin-penetrating substance can be greatly improved. Considering the area of the protrusion and the adhesiveness of the protrusion, the shape of the protrusion may be preferably a cylindrical shape.

In one embodiment of the present invention, the patterned protrusions may have a width of 0.1 mm to 50 mm, preferably 1 mm to 10 mm.

In addition, the height may be 0.01 mm to 1 mm, preferably 0.01 mm to 0.1 mm.

The protrusions of the present invention may include, for example, a nano-sized material as a skin penetrating substance. In order to smoothly administer the skin penetrating substance, it is necessary for the film-type patch to adhere to the skin without lifting for about 8 to 24 hours. Therefore, it is necessary that the height of the protrusions is formed within the above-mentioned range. If the height of the protrusions is too high, the adhesiveness to the skin is reduced, and the administration of the skin penetrating substance may not be performed properly. If the height of the protrusions is too low, the content of the skin penetrating substance may not be sufficient.

In addition, the spacing between the protrusions may be, for example, 0.5 mm to 3 mm, preferably 0.5 mm to 2 mm, and more preferably 0.6 mm to 1.1 mm, but is not limited thereto, and the spacing may be set differently depending on the intended use of the patch.

In the present invention, nano-sized substances and the like can be used as a skin penetrating substance. In order for these substances to penetrate the skin, they must adhere properly to the skin. If the adhesion is not properly achieved, the penetration rate of the skin penetrating substance into the skin will be reduced, making it difficult to expect an effect.

The MTS (Micro-needle Therapy System) patch currently on the market is difficult to produce in large sizes because it is difficult to form needles over a large area. Therefore, the MTS patch is only manufactured for application to very small areas such as wounds or acne.

In addition, the MTS patch has the disadvantage that when it is reattached to the same area after initial attachment and detachment, the skin penetration resistance to the needle and the skin penetrating substance is increased, thereby making attachment difficult and reducing the penetration effect.

That is, there is a disadvantage that when reattached, some of the needles formed on the surface of the skin to which the patch is attached do not insert into the skin, and thus, the skin penetrating substance does not penetrate into the skin. In addition, there is a disadvantage that as described above, the patch is lifted due to the needle not being inserted into the skin, and the patch is easily detached from the skin.

In particular, there is a disadvantage that the space between the needles is prone to lifting, moisture released from the skin remains in this space, and the position of the needles becomes unstable due to movement of the human body, and thus the detachment of the patch from the skin is accelerated.

Meanwhile, in the case of the MTS patch, the needles must be longer than 400 μm to reach the dermis, and when these needles are inserted into the skin, there is a disadvantage that skin pain is significantly increased.

On the other hand, in the case of the patch of the film of the present invention, since the surface of the protrusions that come into contact with the skin is manufactured in a flat shape, it has the advantages of being easy to manufacture on a large scale, having excellent adhesiveness to the skin, and allowing the skin penetrating substance to effectively penetrate the skin.

The film-type patch of the present invention has the characteristic that it may be formed to be 5 cm² or more, 7 cm² or more, 10 cm² or more, 15 cm² or more, 20 cm² or more, 30 cm² or more, 40 cm² or more, or 50 cm² or more.

The patch of the film of the present invention, when attached to a wound, can effectively deliver drugs over a wide area without irritating the wound, unlike conventional acrosome-type needles. For example, when hyaluronic acid, acne prevention or treatment agents, etc. are included in the protrusions, excellent therapeutic effects can be provided because the drugs can be effectively delivered through a large contact area.

In addition, when the protrusions are formed in a columnar shape as described above, the protrusions can be easily formed by a punching process, and thus the number of manufacturing processes can be greatly reduced. Therefore, the manufacturing cost of the patch can be significantly reduced by improving productivity.

In one embodiment of the present invention, the solvent contained in the protrusions may be in the range of 10 to 11 wt. % based on the total weight of the protrusions. If the solvent is contained in the above range, since the production of defective products due to protrusions not being fixed to the adhesive layer and coming off during punching is prevented, excellent production efficiency and quality can be provided.

In the above, if the solvent is less than 10 wt. %, the adhesive strength of the protrusions to the adhesive layer is insufficient, so that the protrusions may easily detach from the adhesive layer during punching, and if the solvent exceeds 11 wt. %, there may be a problem that the protrusions stick to the punching device and are detached from the adhesive layer.

As the solvent, water (e.g., distilled water), lower alcohol, glycerin, etc. may be used, and water may be preferably used. As the water, distilled water may be preferably used.

In one embodiment of the present invention, the patterned protrusions 10 may further comprise a carrier.

In one embodiment of the present invention, the patch 100 may comprise a plurality of protrusions arranged with equal spacing. Additionally, it may comprise one or more groups of protrusions positioned at a distance from each other, and the protrusions comprised in each group may be arranged with equal spacing.

In one embodiment of the present invention, for example, a polyurethane film can be used as the support substrate 30, but is not limited thereto, and any support film known in the art may be used without limitation.

Hereinafter, the composition of the adhesive layer and the protrusion of the present invention will be described in more detail.

### [Adhesive Layer]

In one embodiment of the present invention, the adhesive layer 20 can be manufactured from a material known in the art, and the material is not particularly limited.

The adhesive layer 20 may comprise a synthetic rubber component. Examples of the synthetic rubber include isoprene-styrene polymer (Styrene/Isoprene Copolymer), polydimethylsiloxane, butyl rubber, ethylene-vinyl acetate copolymer, ethylene-ethyl acrylate copolymer, poly(alkyl vinyl ether) (e.g., poly(propyl vinyl ether), poly(isopropyl vinyl ether), poly(butyl vinyl ether) etc.), poly(2-methylpropene), poly(ethylethylene), poly(1,2-dimethylethylene), ethylethylene-1,2-dimethylethylene copolymer, polyisoprene, polybutadiene, styrene-isoprene-styrene block copolymer, styrene-butadiene-styrene block copolymer, etc., and any one of these may be used alone or two or more of these may be used in combination.

The content of the synthetic rubber is not particularly limited, but may be 5 to 50 wt. %, preferably 10 to 30 wt. %, and more preferably 15 to 25 wt. %, based on the total weight of the adhesive layer. If the content of synthetic rubber is less than 5 wt. %, there is a concern that the internal cohesion of the adhesive layer will be reduced, and if the content of synthetic rubber exceeds 50 wt. %, there is a concern that the adhesive layer will become hard and the adhesiveness will be reduced.

The adhesive layer 20 may comprise a tackifier. The tackifier is not particularly limited, and examples thereof may comprise polybutenes, rosin-based resins, terpene-based resins, petroleum-based resins [e.g., petroleum-based aliphatic hydrocarbon resins, petroleum-based aromatic hydrocarbon resins, petroleum-based aliphatic/aromatic copolymer hydrocarbon resins, petroleum-based alicyclic hydrocarbon resins (aromatic hydrocarbon resins that have been hydrogenated), etc.], coumarone-based resins, etc. Among them, petroleum-based alicyclic hydrocarbon resins (aromatic hydrocarbon resins that have been hydrogenated), for example, hydrogenated hydrocarbon (C=6-20) polymers (Hydrogenated Poly (C6-20 Olefin)) are preferable. The tackifier may be used alone or in a combination of two or more.

The content of the tackifier may be 20 to 50 wt. %, preferably 35 to 45 wt. %, based on the total weight of the adhesive layer. If the content of the tackifier is less than 20 wt. %, the adhesiveness may be insufficient, and if the content of the tackifier exceeds 50 wt. %, the adhesive layer may be destroyed, which is not preferable.

The adhesive layer 20 may contain carboxymethyl cellulose. The carboxymethyl cellulose may be contained in an amount of 20 to 45 wt. %, and preferably 25 to 32 wt. %, based on the total weight of the adhesive layer.

The adhesive layer 20 above may contain oils. As the oils, ingredients known in this field may be used, and for example, mineral oils may be preferably used. The oils may be contained in an amount of 5 to 20 wt. %, and more preferably 8 to 13 wt. %, based on the total weight of the adhesive layer.

In addition to the above components, the adhesive layer 20 may further contain additives commonly used in this field. The additives may be contained in an amount of 1 to 10 wt. % based on the total weight of the adhesive layer.

The adhesive layer of the present invention may be manufactured by applying and drying a composition for forming an adhesive layer on a release liner to form an adhesive layer, and laminating a support substrate on the adhesive layer. In addition, the composition for forming the adhesive layer may be directly applied and dried on the support substrate to manufacture the adhesive layer on a support.

### [Patterned Protrusions]

The patterned protrusions can release a drug upon contact with the skin by dissolving or gelling at a certain rate.

The protrusions may comprise a skin penetrating substance, a carrier, and a solvent.

As the skin penetrating substance, a drug that can be administered transdermally can be used. Examples may comprise general anesthetics, hypnotic and sedative drugs, antiepileptics; antipyretic analgesic or anti-inflammatory drugs; anti-vertiginous drugs, psychotropic drugs, central nervous system drugs, anti-dementia drugs, local anesthetics, skeletal muscle relaxants, autonomic nervous system drugs, antispasmodics, anti-Parkinson's drugs, antihistamines, cardiac stimulants, anti-arrhythmic drugs, diuretics, antihypertensives, vasoconstrictors, vasodilators, peripheral vasodilators, anti-arteriosclerotic drugs, medicines for the circulatory system, respiratory stimulants, antitussive expectorants, hormonal drugs, external medicines for purulent diseases; medicines for pain relief, antipruritic, astringent or anti-inflammatory; parasitic skin disease medicine, hemostatic medicine, gout treatment medicine, diabetes medicine, anti-malignant tumor medicine, antibiotics, chemotherapy drugs, narcotics, hyaluronic acid, acne prevention and treatment agents, smoking cessation aids, etc.

In addition, the above-described drugs comprise not only free base drugs but also physiologically acceptable salts thereof. The salts are not particularly limited, but include, for example, formate, acetate, lactate, adipate, citrate, tartrate, methanesulfonate, fumarate, maleate, etc. and as addition salts of inorganic acids, hydrochloride, sulfate, nitrate, phosphate, etc. can be exemplified. In addition, the drug may be a solvate, and may be a hydrate or non-hydrate.

Specifically, hyaluronic acid, acne prevention and treatment agents, etc. can be used.

As a drug carrier, a component that is gelled by a solvent released from the skin and can release the drug contained in the drug carrier due to the gelation of the drug support layer can be used. For example, one selected from the group consisting of hyaluronic acid, polyvinylpyrrolidone (PVP), polyvinyl alcohol (PVA), sodium carboxymethyl cellulose (NaCMC), poloxamer, carbomer, hypromellose, hydroxypropyl cellulose (HPC), hydroxyethyl cellulose (HEC), sodium alginate, saccharides, glycerin, propylene glycol, polyethylene glycol 400, and sorbitol (SB) can be used alone, or a combination of two or more of these can be used.

The saccharide may be at least one selected from the group consisting of a monosaccharide, a disaccharide, and a polysaccharide, and more specifically, the monosaccharide may be at least one selected from the group consisting of fructose, galactose, glucose, and mannose; the disaccharide may be at least one selected from the group consisting of sucrose, lactose, maltose, trehalose, turanose, and cellobiose; and the polysaccharide may be at least one selected from the group consisting of dextran, diethylamino ethyldextran, dextrin, cellulose, and β-glucans.

As the solvent, water (e.g., distilled water), lower alcohol, glycerin, etc. may be used, and water may be preferably used. As the water, distilled water may be preferably used.

In one embodiment of the present invention, the protrusion may comprise 0.1 to 20 wt. % of a skin penetrating substance, 69 to 89 wt. % of a carrier, and 10 to 11 wt. % of a solvent. In addition, it may further comprise 0.1 to 10 wt. % of a functional additive and/or a common additive known in the art.

In the above, the structure of the patch of the present invention has been described, but it should not be understood that the patch according to the present invention comprises only the components described above. Although the description is omitted in this specification, it should be understood that components obvious to those skilled in the art may be additionally comprised in the patch of the present invention.

Hereinafter, a method of manufacturing a film-type patch of the present invention will be described by way of example.

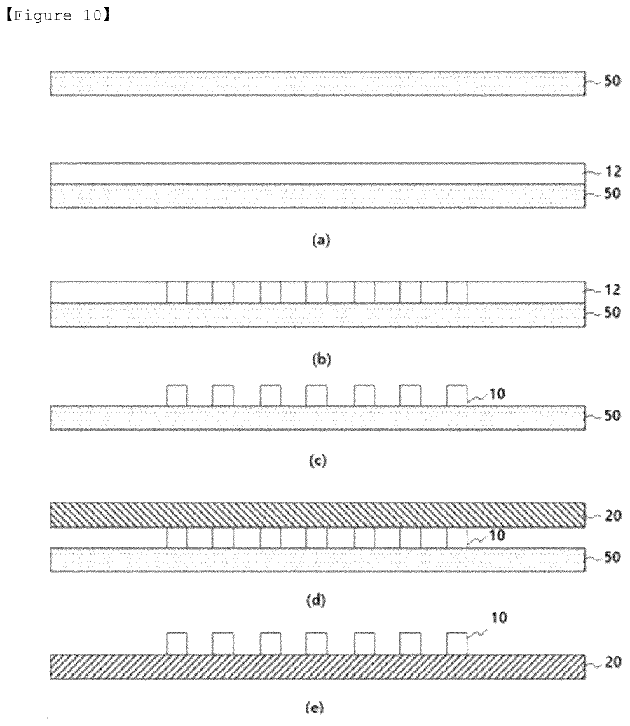

The film-type patch of the present invention, as shown in FIG. 8, may be manufactured by a method comprising the steps of:
a) laminating a protrusion forming sheet 12 on one side of an adhesive sheet 20;
b) punching out a plurality of patterned protrusions into the protrusion forming sheet 12; and
c) removing the protrusions forming sheet 12 excluding the protrusions patterned by punching from the adhesive sheet 20,
wherein before or after the step (a), or before or after step (c), a support substrate 30 is laminated on the other side of the adhesive sheet 20, and then the adhesive sheet and the support substrate located in the space between the protrusions 10 are perforated to form a plurality of holes.

The patterned protrusions may comprise a carrier with a skin penetrating substance.

The protrusion forming sheet may have a solvent content of 10 to 11 wt. %.

The contents described in the film-type patch above can be commonly applied to all methods of manufacturing the present invention, so any duplicated contents are omitted below.

Additionally, the components of the protrusion forming sheet are identical to the components of the protrusion described above.

The method of manufacturing the patch of the present invention has the characteristic of significantly reducing the number of manufacturing processes compared to the conventional technology, so that the manufacturing cost of the patch may be significantly reduced.

The protrusion forming sheet may be manufactured into a sheet shape by a method known in the art.

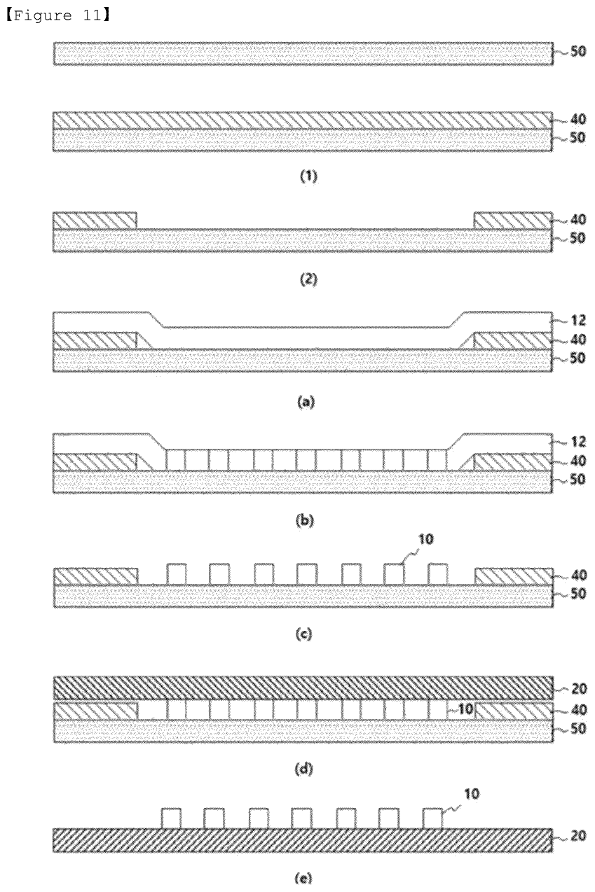

In addition, the film-type patch of the present invention,
before laminating the protrusion forming sheet on one side of the adhesive sheet in the step a), further comprises the steps of:
1) laminating the releasing film 40 on one side of the adhesive sheet 20; and
2) forming a hollow portion by punching out the center of the releasing film 40 with an area for arranging protrusions and removing the punched center of the releasing film 40, as illustrated in FIG. 9,
wherein the protrusion forming sheet 12 is laminated to cover the hollow portion and the remaining releasing film,
the step of punching out a plurality of patterned protrusions into the protrusion forming sheet in the step b) is performed on the protrusion forming sheet placed in the hollow portion, and
in the step c), when removing the protrusions forming sheet 12 excluding the protrusions patterned by punching from the adhesive sheet 20, the releasing film 40 is also removed.

In another embodiment of the present invention, the film-type patch of the present invention may be manufactured by a method comprising the steps of:
a) laminating a protrusion forming sheet 12 on one side of a first adhesive sheet 50;
b) punching out a plurality of patterned protrusions 12 on the protrusion forming sheet 12;
c) removing the protrusions forming sheet 12 excluding the protrusions 10 patterned by punching from the first adhesive sheet 50;
d) laminating a second adhesive sheet 20 on the upper surface of the patterned protrusions 10; and
e) separating the second adhesive sheet 20 and the protrusions 10 adhered thereto from the first adhesive sheet 50, as shown in FIG. 10,
wherein, before or after the step d), or after step (e), a support substrate is laminated on the other side of the second adhesive sheet, and then the adhesive sheet and the support substrate located in the space between the protrusions are perforated to form a plurality of holes.

The patterned protrusions may comprise a carrier with a skin penetrating substance.

The protrusion forming sheet may have a solvent content of 10 to 11 wt. %.

In the manufacturing method, the adhesive strength of the second adhesive sheet to the protrusions may be greater than the adhesive strength of the first adhesive sheet to the protrusions.

In one embodiment of the present invention, as shown in FIG. 11,
the method, before laminating the protrusion forming sheet 12 on one side of the first adhesive sheet 50 in the step a), further comprises the steps of:
1) laminating the releasing film 40 on one side of the first adhesive sheet 50; and
2) forming a hollow portion by punching out the center of the releasing film 40 with an area for arranging protrusions and removing the punched center of the releasing film 40,
wherein the protrusion forming sheet 12 is laminated to cover the hollow portion and the remaining releasing film 40, and
the step of punching out a plurality of patterned protrusions 10 into the protrusion forming sheet 12 in the step b) can be performed on the protrusion forming sheet arranged in the hollow portion.

Hereinafter, the present invention will be described in detail by way of examples in order to specifically explain the present invention. However, the embodiments according to the present invention can be modified in various different forms, and the scope of the present invention should not be construed as being limited to the embodiments described below. The embodiments of the present invention are provided to more completely explain the present invention to a person having average knowledge in the art.

### Example 1: Manufacturing of Film-Type Patch

### (1) Manufacturing of Protrusion Forming Sheet

5 wt % of hyaluronic acid as a drug, 1.2 wt % of hydrolyzed collagen, 50 wt % of polyvinylpyrrolidone (PVP) as a drug carrier, 28.5 wt % of polyvinyl alcohol, 2.1 wt % of pulluran, 1.2 wt % of PEG-60 hydrogenated castor oil, 1.5 wt % of sodium chloride, and 10.5 wt % of distilled water as a solvent were mixed to prepare a composition for a protrusion forming sheet. The composition was applied onto a polyethylene terephthalate (PET) liner (thickness: 75 μm) that had undergone silicone release treatment to manufacture a protrusion forming sheet having a thickness of 40 μm.

### (2) Manufacturing of Adhesive Sheet

43 wt. % of hydrogenated (C=6-20) poly (C6-20 olefin), 18 wt. % of isoprene-styrene polymer (Styrene/Isoprene Copolymer), 28 wt. % of carboxymethyl cellulose, and 11 wt. % of mineral oil were mixed to obtain an adhesive component.

The adhesive component was applied onto a polyethylene terephthalate (PET) liner (thickness 75 μm) that had undergone silicone peeling treatment so that the thickness became 300 μm after drying, and a moisture-permeable, waterproof polyurethane film was laminated to manufacture the adhesive sheet.

### (3) Manufacturing of Film-Type Patch

The adhesive sheet manufactured in (2) above was laminated on a polyurethane support substrate.

Next, the protrusion forming sheet manufactured in (1) above was laminated on one side of the adhesive sheet.

The protrusion forming sheet was punched using a puncher with a diameter of 1.25 mm.

The remaining protrusion forming sheet, excluding the protrusions patterned by punching from the adhesive sheet, was removed from the adhesive layer.

After this, 300 holes were perforated through the adhesive sheet and the support substrate using a perforator at room temperature in the space formed between one protrusion and the adjacent other protrusions and in the area where no protrusions were located, and thus overall, holes were formed between protrusions and on the outer periphery of the patch where no protrusions were formed.

After this, a release paper was placed over the adhesive sheet and the patterned protrusions, placed in packaging paper, and sealed to manufacture a film-type patch.

### Example 2: Manufacturing of Film-Type Patch

A film-type patch was manufactured in the same manner as in Example 1, except that the amount of polyvinyl alcohol used in manufacturing the protrusion forming sheet in the Example 1 is reduced to 28 wt. % and the amount of distilled water used is increased to 11 wt. %.

### Example 3: Manufacturing of Film-Type Patch

A film-type patch was manufactured in the same manner as in Example 1, except that when perforating holes penetrating the adhesive sheet and the support substrate in the Example 1, the patch is cooled to 5° C. and a perforator is used to form the holes.

### Example 4: Manufacturing of Film-Type Patch

A film-type patch was manufactured in the same manner as in Example 1, except that a picosecond laser with a pulse width of 1 ps is used to punch holes penetrating the adhesive sheet and the support substrate in the Example 1.

### Example 5: Manufacturing of Film-Type Patch

A film-type patch was manufactured in the same manner as in Example 1, except that in the Example 1, before punching holes penetrating the adhesive sheet and the support substrate, the releasing film is further laminated on the surface where the adhesive layer and the plurality of patterned protrusions are exposed, and then 300 holes penetrating the support substrate, the adhesive sheet, and the releasing film are punched.

### Comparative Example 1: Manufacturing of Film-Type Patch

A film-type patch was manufactured using the same method as in Example 1, except that in the Example 1, a hole penetrating the adhesive sheet and the support substrate is not formed when manufacturing the protrusion forming sheet.

### Comparative Example 2: Manufacturing of Film-Type Patch

A film-type patch was manufactured using the same method as in Example 1, except that in the Example 1, when manufacturing the protrusion forming sheet, the holes penetrating the adhesive sheet and the support substrate are not formed, but the same number of holes as in Example 1 are formed only on the support substrate.

### Comparative Example 3: Manufacturing of Film-Type Patch

A film-type patch was manufactured using the same method as in Example 1, except that in the Example 1, when manufacturing a protrusion forming sheet, the amount of polyvinyl alcohol used is increased to 29.7 wt. %, and the amount of distilled water used is reduced to 9.3 wt. %.

### Comparative Example 4: Manufacturing of Film-Type Patch

A film-type patch was manufactured using the same method as in Example 1, except that in the Example 1, when manufacturing a protrusion forming sheet, the amount of polyvinyl alcohol used is reduced to 27.8 wt. %, and the amount of distilled water used is increased to 11.2 wt. %.

### Test Example 1: Evaluation of Skin Adhesive Force of Film-Type Patch

The patches manufactured in the Example 1 and Comparative Examples 1 and 2 were attached to the neck area and observed for 12 hours.

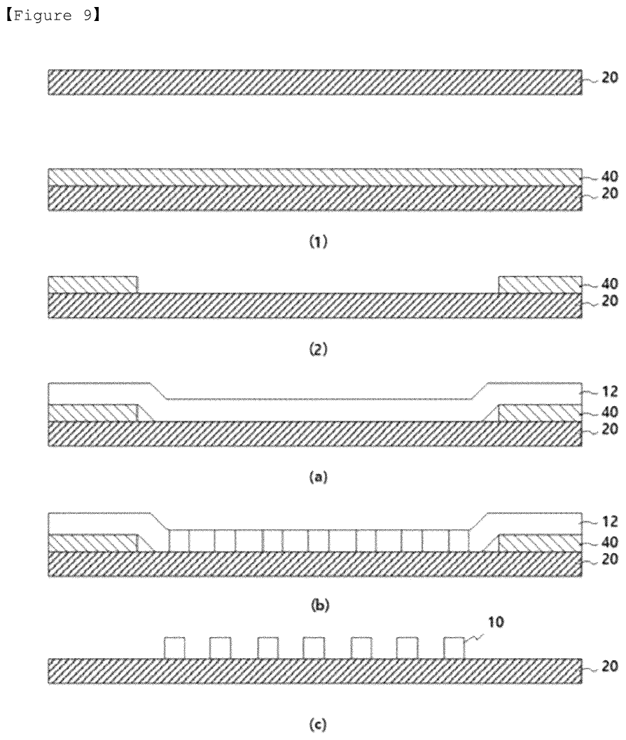

The test results are shown in Table 1, FIGS. 6 and 7 below.

TABLE 1

| | At initial | 12 hours after |
|---|---|---|
| Film-type patch | attachment | attachment |
| Example 1 | no lifting | no lifting |
| Comparative Example 1 | 15% lifting | 30% lifting |
| Comparative Example 2 | 10% lifting | 25% lifting |

From the results in Table 1 and FIGS. 6 and 7 above, it can be confirmed that in the case of the patch of Example 1, which formed a plurality of holes penetrating the adhesive sheet and the support substrate, the skin adhesive force was significantly improved compared to Comparative Examples 1 and 2, which did not form such holes. In addition, it can be confirmed that in the case where the holes are formed only in the support substrate (Comparative Example 2), a slightly better effect is shown compared to the case where holes are not formed (Comparative Example 1), but the difference in effect is not very large compared to Example 1 of the present invention.

### Test Example 2: Evaluation of Quality of Film-Type Patch

The morphology of the patches manufactured in the Examples 1 and 2 and Comparative Examples 3 and 4 was photographed by a camera and evaluated.

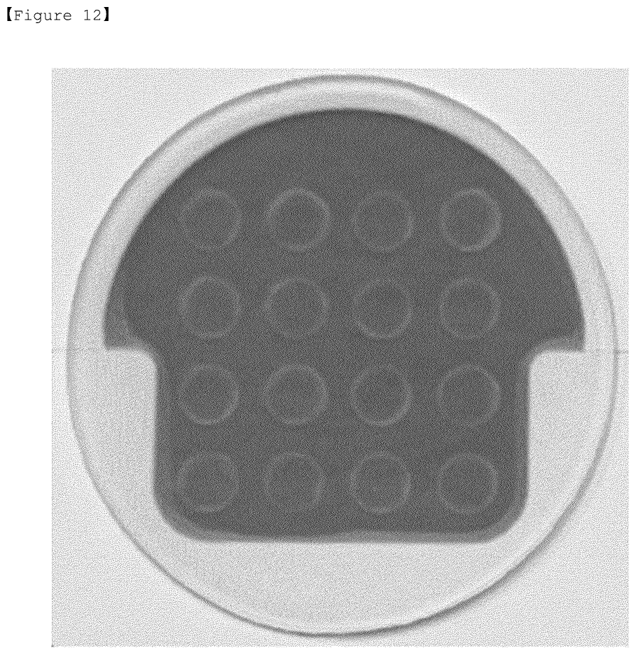

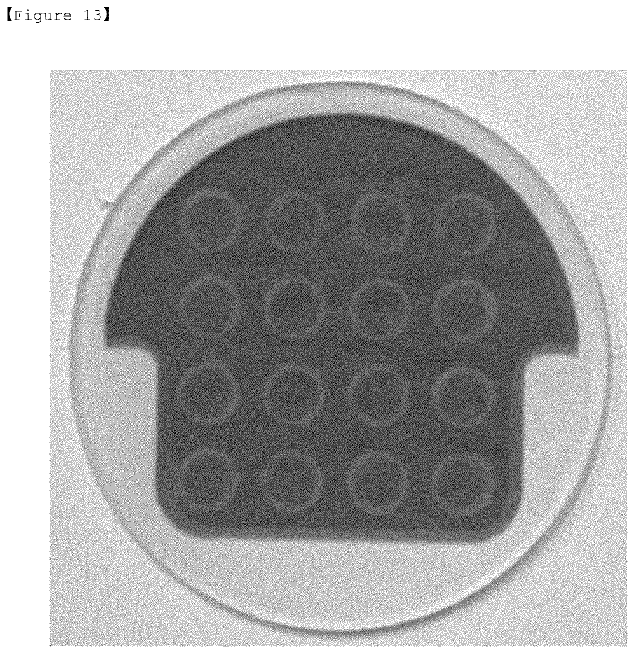

The results of the evaluation are shown in FIGS. 12 to 15. FIG. 12 is a photograph of a patch of Example 1 of the present invention, and FIG. 13 is a photograph of a patch of Example 2 of the present invention. As can be seen from FIGS. 12 and 13, it can be seen that patches of Examples 1 and 2 of the present invention were manufactured as patches of excellent quality.

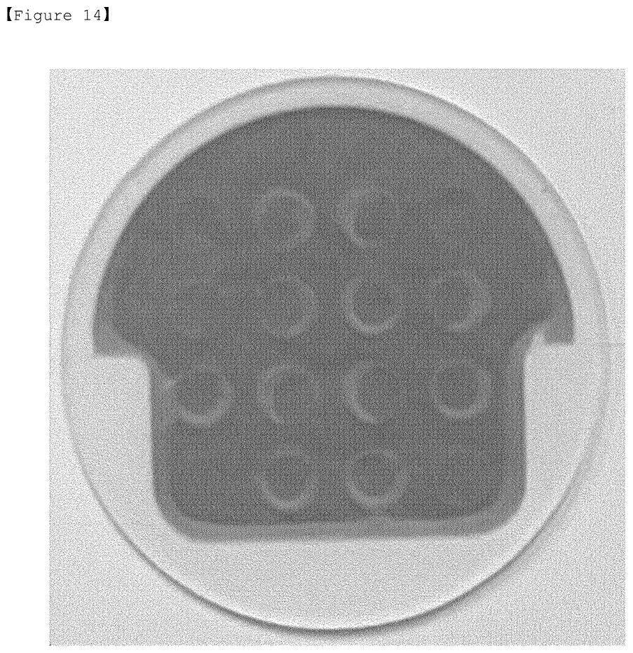

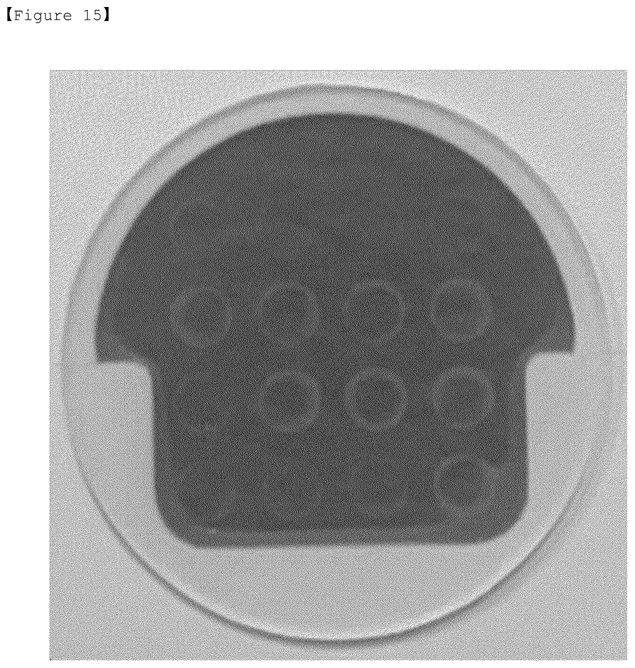

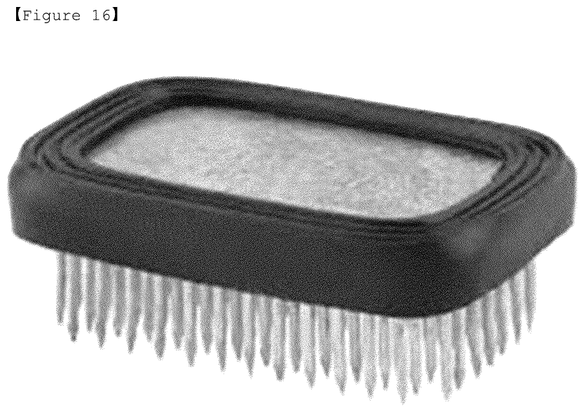

Meanwhile, FIG. 14 is a photograph of a patch of Comparative Example 3, and FIG. 15 is a photograph of a patch of Comparative Example 4. As can be seen from the FIGS. 14 and 15, it can be seen that in the case of patches of Comparative Examples 3 and 4, a number of protrusions are detached from the adhesive layer during punching, resulting in the production of defective products.

Therefore, it can be seen from the above experiment that the protrusions should contain a solvent in the range of 10 to 11 wt. %, and if the solvent content is outside the above range, the protrusions are not fixed to the adhesive layer and are detached during punching, resulting in the production of defective products.

Likewise, the protrusion forming sheet should also contain solvent in the range of 10 to 11 wt. %.

In the above, although the preferred examples of the present invention have been described in detail, the scope of the present invention is not limited thereto, and various modifications and improvements made by those skilled in the art using the basic concept of the present invention defined in the following claims also fall within the scope of the present invention.

[Description of the symbol]

| | |
|---|---|
| 10: protrusions | 12: protrusion forming sheet |
| 20: adhesive layer (second adhesive layer) | |
| 30: support substrate | |
| 40: releasing film | 50: first adhesive layer |
| 60: hole | 70: releasing film |
| 100: patch | |

1. A film-type patch, comprising:
a support layer,
an adhesive layer laminated on an upper surface of the support layer, and
a plurality of patterned protrusions spaced apart from each other and attached to an upper surface of the adhesive layer,
wherein the patterned protrusions contain a skin penetrating substance,
space between the protrusions is provided with a number of holes penetrating the support layer and the adhesive layer, and
the patterned protrusions dissolve or gel at a certain rate when in contact with the skin.

2. The film-type patch according to claim 1, wherein the holes are provided in 2 to 8 pieces in the space formed between one protrusion and adjacent other protrusions.

3. The film-type patch according to claim 1, wherein the hole has a longitudinal length of 0.1 mm to 2.5 mm.

4. The film-type patch according to claim 3, wherein a plurality of holes penetrating the support layer and the adhesive layer are further formed in a portion of the entire are of the film-type patch where the protrusions are not formed.

5. The film-type patch according to claim 1, wherein the hole is formed using a perforator.

6. The film-type patch according to claim 1, wherein the patterned protrusions have a width of 0.1 mm to 50 mm and a height of 0.01 mm to 1 mm.

7. The film-type patch according to claim 1, wherein the protrusions are in the shape of cylinders, elliptical columns, or polygonal columns.

8. The film-type patch according to claim 1, wherein the film-type patch further comprises a releasing film laminated on the surface where the adhesive layer and the patterned protrusions are exposed.

9. The film-type patch according to claim 8, wherein the film-type patch has a plurality of holes formed penetrating the support layer, the adhesive layer, and the releasing film.

10. The film-type patch according to claim 1, wherein the protrusions have a solvent content of 10 to 11 wt. %.

11. The film-type patch according to claim 10, wherein the solvent is at least one selected from water, lower alcohol, and glycerin.

12. The film-type patch according to claim 1, wherein the skin penetrating substance is hyaluronic acid.

13. The film-type patch according to claim 1, wherein the protrusions further comprises at least one carrier selected from the group consisting of hyaluronic acid, polyvinylpyrrolidone (PVP), polyvinyl alcohol (PVA), sodium carboxymethyl cellulose (NaCMC), poloxamer, carbomer, hypromellose, hydroxypropyl cellulose (HPC), hydroxyethyl cellulose (HEC), sodium alginate, saccharides, glycerin, propylene glycol, polyethylene glycol 400, and sorbitol (SB).

14. A method of manufacturing a film-type patch according to claim 1 comprising the steps of:
a) laminating a protrusion forming sheet on one side of an adhesive sheet;
b) punching out a plurality of patterned protrusions into the protrusion forming sheet; and
c) removing the protrusion forming sheet excluding the protrusions patterned by punching from the adhesive sheet,
wherein before or after the step (a), or before or after step (c), a support substrate is laminated on the other side of the adhesive sheet, and then the adhesive sheet and the support substrate located in the space between the protrusions are perforated to form a plurality of holes.

15. The method of manufacturing a film-type patch according to claim 14, wherein the protrusion forming sheet has a solvent content of 10 to 11 wt. %.

16. The method of manufacturing a film-type patch according to claim 14, wherein the method, before laminating the protrusion forming sheet on one side of the adhesive sheet in the step a), further comprises the steps of:
1) laminating the releasing film on one side of the adhesive sheet; and
2) forming a hollow portion by punching out the center of the releasing film with an area for arranging protrusions and removing the punched center of the releasing film,
wherein the protrusion forming sheet is laminated to cover the hollow portion and the remaining releasing film,
in the step b), the step of punching out a plurality of patterned protrusions into the protrusion forming sheet is performed on the protrusion forming sheet placed in the hollow portion, and
in the step c), when removing the protrusion forming sheet excluding the protrusions patterned by punching from the adhesive sheet, the releasing film is also removed.

17. A method of manufacturing a film-type patch according to claim 1, comprising the steps of:
a) laminating a protrusion forming sheet on one side of a first adhesive sheet;
b) punching out a plurality of patterned protrusions into the protrusion forming sheet;
c) removing the protrusion forming sheet excluding the protrusions patterned by punching from the first adhesive sheet;
d) laminating a second adhesive sheet on the upper surface of the patterned protrusions; and
e) separating the second adhesive sheet and the protrusions adhered thereto from the first adhesive sheet,
wherein, before or after the step d), or after step (e), a support substrate is laminated on the other side of the second adhesive sheet, and then the adhesive sheet and the support substrate located in the space between the protrusions are perforated to form a plurality of holes.

18. The method of manufacturing a film-type patch according to claim 17, wherein the protrusion forming sheet has a solvent content of 10 to 11 wt. %.

19. The method of manufacturing a film-type patch according to claim 17, wherein the method, before laminating the protrusion forming sheet on one side of the first adhesive sheet step a), further comprises the steps of:
1) laminating the releasing film on one side of the first adhesive sheet; and
2) forming a hollow portion by punching out the center of the releasing film with an area for arranging protrusions and removing the punched center of the releasing film,
wherein the protrusion forming sheet is laminated to cover the hollow portion and the remaining releasing film, and
in the step b), the step of punching out a plurality of patterned protrusions into the protrusion forming sheet is performed on the protrusion forming sheet arranged in the hollow portion.
